(19) United States
(12) Patent Application Publication
(10) Pub. No.: US 2021/0095243 A1
(43) Pub. Date: Apr. 1, 2021

(54) GENOME-WIDE RATIONALLY-DESIGNED MUTATIONS LEADING TO ENHANCED TYROSINE PRODUCTION IN S. CEREVISIAE

(71) Applicant: Inscripta, Inc., Boulder, CO (US)

(72) Inventors: Bryan Leland, Boulder, CO (US); Michael Clay, Boulder, CO (US); Eric Abbate, Boulder, CO (US); Stephen Federowicz, Boulder, CO (US); Richard Fox, Boulder, CO (US)

(21) Appl. No.: 17/026,359

(22) Filed: Sep. 21, 2020

Related U.S. Application Data
(60) Provisional application No. 62/907,402, filed on Sep. 27, 2019.
(60) Provisional application No. 62/912,544, filed on Oct. 8, 2019.

(51) Int. Cl. C12N 1/14; C12N 15/10; C12N 9/10

(52) U.S. Cl. CPC C12N 1/14; C12Y 205/01054; C12N 9/1085; C12N 15/102

(57) ABSTRACT

The present disclosure relates to various different types of mutations in S. cerevisiae leading to enhanced tyrosine production for, e.g., supplements and nutraceuticals.

### RELATED APPLICATIONS

The present application claims the benefit of priority to U.S. Provisional Application Nos. 62/907,402 filed Sep. 27, 2019, and 62/912,544 filed Oct. 8, 2019 and are incorporated herein by reference.

### FIELD OF THE INVENTION

The present disclosure relates to mutations in genes in S. cerevisiae leading to enhanced tyrosine production.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The amino acid tyrosine is an α-amino acid that is one of the twenty standard amino acids that are used by cells to synthesize proteins. Tyrosine is a non-essential amino acid with a polar side group and often occurs in proteins that are part of signal transduction processes, functioning as a receiver of phosphate groups that are transferred by way of protein kinases. L-tyrosine and its derivatives (L-DOPA, melanin, phenylpropanoids and others) are used in pharmaceuticals, dietary supplements and food additives. Because of these roles as, e.g., a supplement and nutraceutical, there has been a growing effort to produce tyrosine on a large scale.

Accordingly, there is a need in the art for organisms—specifically genetically engineered organisms—that produce enhanced amounts of tyrosine where such organisms can be harnessed for large scale tyrosine production. The disclosed nucleic acid sequences from S. cerevisiae satisfy this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

Mutagenesis libraries specifically targeting the genes the tyrosine/shikimate pathway were designed for saturation mutagenesis. Additionally, to more deeply explore the rest of the S. cerevisiae genome for new targets involved in tyrosine biosynthesis, libraries were designed to target all annotated loci with either premature stop codons (for a knock-out phenotype) or with an insertion of a set of five synthetic terminator variants (for expression modulation phenotypes). The present disclosure thus provides mutant S. cerevisiae APO4 sequences and other mutants that produce enhanced amounts of tyrosine in culture. S. cerevisiae APO4 is also known as 3-deoxy-D-arbino-heptulosonate-7-phosphate (DAHP) synthase, which is the first enzyme in a series of metabolic reactions known as the shikimate pathway. The shikimate pathway is responsible for the biosynthesis of the amino acids phenylalanine, tyrosine and tryptophan. Thus, in some embodiments, the present disclosure provides any one of SEQ ID Nos. 1-100.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A shows several residues important to Tyrosine production that are located near the Tyrosine allosteric binding site in the ARO4 protein. ARO4 is colored green, residues important for Tyrosine production are orange (with residue number identified) and Tyrosine is in cyan. FIG. 1B shows residues important to Tyrosine production that are localized near the phosphoenolpyruvate (PEP) substrate binding site in the ARO4 protein. ARO4 is colored green, residues important for Tyrosine production are colored blue (with residue number identified) and PEP is magenta.

FIG. 2A is a simplified structure of the coding sequence for the LexA-Rad51 fusion protein that aids in enhancing homologous recombination. FIG. 2B is a simplified graphic of enhancing homologous recombination—and thus increasing editing efficiency—using the LexA-Rad51 fusion protein. FIG. 2C is an exemplary vector map comprising a coding sequence for the LexA-Rad51 fusion protein, an editing or “CREATE” cassette comprising the edits for enhancing tyrosine production, and the coding sequence for the nuclease MAD7.

It should be understood that the drawings are not necessarily to scale, and that like reference numbers refer to like features.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; and Chang, et al., Guide to Electroporation and Electrofusion, Academic Press, California (1992), all of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The term “CREATE cassette” or “editing cassette” refers to a gRNA linked to a donor DNA or HA. The term “CREATE cassette array” or “editing cassette array” refers to two or more linked CREATE cassettes. For information on CREATE cassettes, please see U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,185, issued 5 Nov. 2019; U.S. Pat. No. 10,519,437, issued 31 Dec. 2019; U.S. Pat. No. 10,584,333, issued 10 Mar. 2020; U.S. Pat. No. 10,584,334, issued 10 Mar. 2020; U.S. Pat. No. 10,647,982, issued 12 May 2020; U.S. Pat. No. 10,689,645, issued 23 Jun. 2020; U.S. Pat. No. 10,738,301, issued 11 Aug. 2020; and U.S. Ser. No. 16/920,853, filed 6 Jul. 2020; and Ser. No. 16/988,694, filed 9 Aug. 2020.

As used herein the term “donor DNA” or “donor nucleic acid” refers to nucleic acid that is designed to introduce a DNA sequence modification (insertion, deletion, substitution) into a locus (e.g., a target genomic DNA sequence or cellular target sequence) by homologous recombination using nucleic acid-guided nucleases. For homology-directed repair, the donor DNA must have sufficient homology to the regions flanking the “cut site” or site to be edited in the genomic target sequence. The length of the homology arm(s) will depend on, e.g., the type and size of the modification being made. In many instances and preferably, the donor DNA will have two regions of sequence homology (e.g., two homology arms) to the genomic target locus. Preferably, an “insert” region or “DNA sequence modification” region—the nucleic acid modification that one desires to be introduced into a genome target locus in a cell—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the genomic target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the genomic target sequence.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

As used herein, the terms “protein” and “polypeptide” are used interchangeably. Proteins may or may not be made up entirely of amino acids.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible, and in some embodiments the transcription of at least one component of the nucleic acid-guided nuclease editing system is—and often at least three components of the nucleic acid-guided nuclease editing system are—under the control of an inducible promoter. A number of gene regulation control systems have been developed for the controlled expression of genes in plant, microbe, and animal cells, including mammalian cells, including the pL promoter (induced by heat inactivation of the CI857 repressor), the pPhIF promoter (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, Calif.); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, nourseothricin N-acetyl transferase, chloramphenicol, erythromycin, tetracycline, gentamicin, bleomycin, streptomycin, rifampicin, puromycin, hygromycin, blasticidin, and G418 may be employed. In other embodiments, selectable markers include, but are not limited to sugars such as rhamnose. “Selective medium” as used herein refers to cell growth medium to which has been added a chemical compound or biological moiety that selects for or against selectable markers.

The term “specifically binds” as used herein includes an interaction between two molecules, e.g., an engineered peptide antigen and a binding target, with a binding affinity represented by a dissociation constant of about 10⁻⁷ M, about 10⁻⁸ M, about 10⁻⁹ M, about 10⁻¹⁰ M, about 10⁻¹¹ M, about 10⁻¹² M, about 10⁻¹³ M, about 10⁻¹⁴ M or about 10⁻15 M.

The terms “target genomic DNA sequence”, “cellular target sequence”, or “genomic target locus” refer to any locus in vitro or in vivo, or in a nucleic acid (e.g., genome) of a cell or population of cells, in which a change of at least one nucleotide is desired using a nucleic acid-guided nuclease editing system. The cellular target sequence can be a genomic locus or extrachromosomal locus.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. As used herein, the phrase “engine vector” comprises a coding sequence for a nuclease to be used in the nucleic acid-guided nuclease systems and methods of the present disclosure. As used herein the phrase “editing vector” comprises a donor nucleic acid, optionally including an alteration to the cellular target sequence that prevents nuclease binding at a PAM or spacer in the cellular target sequence after editing has taken place, and a coding sequence for a gRNA. The editing vector may also and preferably does comprise a selectable marker and/or a barcode. In some embodiments, the engine vector and editing vector may be combined; that is, all editing and selection components may be found on a single vector. Further, the engine and editing vectors comprise control sequences operably linked to, e.g., the nuclease coding sequence, recombineering system coding sequences (if present), donor nucleic acid, guide nucleic acid(s), and selectable marker(s).

### Library Design Strategy and Nuclease-Directed Genome Editing

Tyrosine or 4-hydroxyphenylalanine is one of the twenty standard amino acids that are used by cells to synthesize proteins. Tyrosine is a non-essential amino acid with a polar side group. While tyrosine is generally classified as a hydrophobic amino acid, it is more hydrophilic than phenylalanine. Aside from being a proteinogenic amino acid, tyrosine has a special role by virtue of its phenol group.

In plants and most microorganisms, tyrosine is produced via prephenate, an intermediate on the shikimate pathway. Prephenate is oxidatively decarboxylated with retention of the hydroxyl group to give p-hydroxyphenylpyruvate, which is transaminated using glutamate as the nitrogen source to produce tyrosine and α-ketoglutarate. Tyrosine occurs in proteins that are part of signal transduction processes and functions as a receiver of phosphate groups transferred by way of protein kinases. A tyrosine residue also plays an important role in photosynthesis. In chloroplasts (photosystem II), tyrosine acts as an electron donor in the reduction of oxidized chlorophyll. In this process, tyrosine loses the hydrogen atom of its phenolic OH-group, where this radical is subsequently reduced in the photosystem II by four core manganese clusters.

In dopaminergic cells in the brain, tyrosine is converted to L-DOPA by the enzyme tyrosine hydroxylase. Tyrosine hydroxylase is the rate-limiting enzyme involved in the synthesis of the neurotransmitter dopamine. Dopamine can then be converted into other catecholamines, such as norepinephrine (noradrenaline) and epinephrine (adrenaline). Tyrosine increases plasma neurotransmitter levels—particularly dopamine and norepinephrine—but has little if any effect on mood in normal subjects; instead, the effect on mood is noted in humans subjected to stressful conditions. A number of studies have found tyrosine to be useful during conditions of stress, cold, fatigue, prolonged work and sleep deprivation, with reductions in stress hormone levels, reductions in stress-induced weight loss seen in animal trials, and improvements in cognitive and physical performance seen in human trials. Tyrosine does not seem to have any significant effect on cognitive or physical performance in normal circumstances but does help sustain working memory better during multitasking. Additionally, the thyroid hormones triiodothyronine (T3) and thyroxine (T4) in the colloid of the thyroid are derived from tyrosine.

L-tyrosine and its derivatives (L-DOPA, melanin, phenylpropanoids, and others) are used in pharmaceuticals, dietary supplements, and food additives. Two methods were formerly used to manufacture L-tyrosine. The first involves the extraction of the desired amino acid from protein hydrolysates (e.g., from plants) using a chemical approach. The second utilizes enzymatic synthesis from phenolics, pyruvate, and ammonia through the use of tyrosine phenol-lyase. Advances in genetic engineering and the advent of industrial fermentation have shifted the synthesis of L-tyrosine to the use of engineered strains of E. coli and other microorganisms.

3-Deoxy-D-arabino-heptulosonate-7-phosphate (DAHP) synthase (ARO4 in yeast) is the first enzyme in a series of metabolic reactions known as the shikimate pathway, which is responsible for the biosynthesis of the amino acids phenylalanine, tyrosine and tryptophan. The primary function of DAHP synthase is to catalyze the reaction of phosphoenolpyruvate and D-erythrose 4-phosphate to DAHP and phosphate. The shikimate pathway is a seven-step metabolic route used by bacteria, archaea, fungi, algae, and some protozoans and plants for the biosynthesis of folates and the aromatic amino acids. This pathway is not found in animals and humans; however, animals and humans require these amino acids, and as such, the products of this pathway represent essential amino acids that must be obtained from organisms which are not animals, or from animals whose diet includes lower organisms who do have the shikimate pathway.

In the studies herein, mutagenesis libraries specifically targeting genes in the shikimate pathway were designed for saturation mutagenesis. Additionally, to more deeply explore the rest of the genome for new targets involved in tyrosine biosynthesis, libraries were designed to target all annotated loci with either premature stop codons (for a knock-out phenotype) or insertion of a set of five synthetic promoter variants (for expression modulation phenotypes).

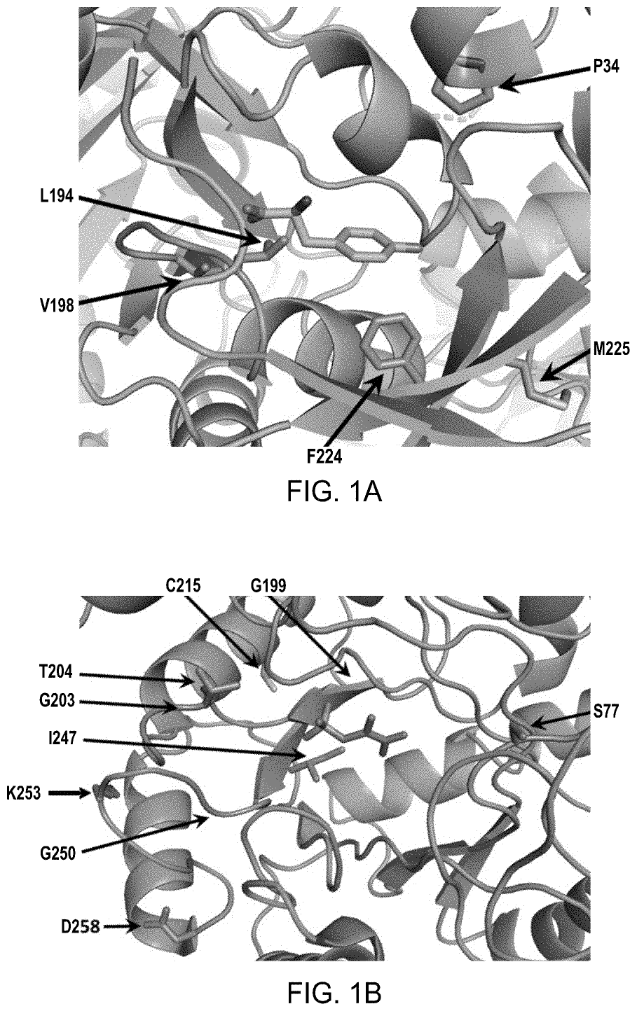

FIG. 1A shows several residues important to Tyrosine production that are located near the Tyrosine allosteric binding site in the ARO4 protein. ARO4 is colored green, residues important for Tyrosine production are colored orange (with residue number identified) and Tyrosine is in cyan. FIG. 1B shows residues important to Tyrosine production that are localized near the phosphoenolpyruvate (PEP) substrate binding site in the ARO4 protein. ARO4 is colored green, residues important for Tyrosine production are colored blue (with residue number identified) and PEP is in magenta.

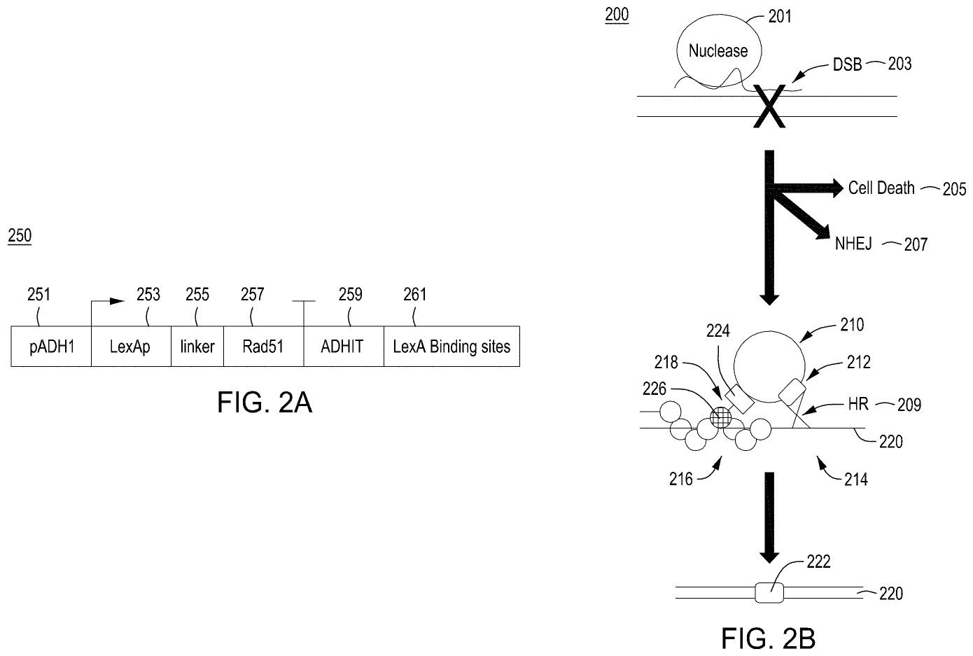

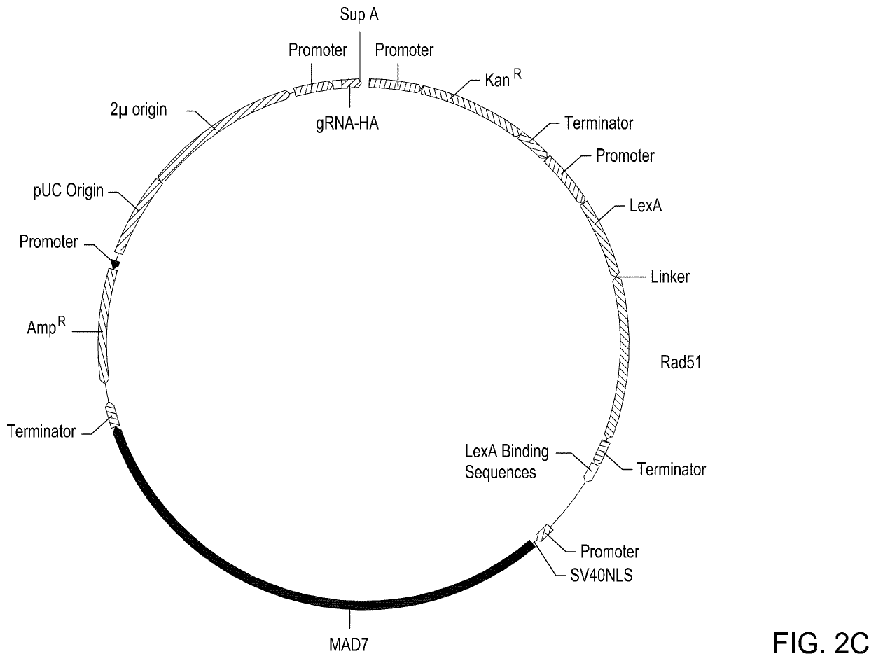

FIG. 2A is a simplified structure 250 of a coding sequence for the LexA-Rad51 fusion protein and a LexA DNA binding domain that forms part of an editing vector (see FIG. 2C) for delivering desired edits to the S. cerevisiae genome. The LexA-Rad51 fusion protein and a LexA DNA binding domain system has been found to significantly increase editing efficiency in S. cerevisiae, and thus was employed in the methods described herein. The components include, from 5′ to 3′, the pADH1 (yeast alcohol dehydrogenase 1) promoter 251; the LexA portion of the LexA-Rad51 fusion protein 253; a linker 255; the Rad51 portion of the LexA-Rad51 fusion protein 257; the ADH1 terminator 259; and a LexA DNA binding site 261.

As an alternative to the yeast alcohol dehydrogenase 1 promoter, other promoters such as pGPD, pTEF1, pACT1, pRNR2, pCYC1, pTEF2, pHXT7, pYEF3, pRPL3, pRPL4 or pGAL1 in a Zev system may be used. The LexA portion of the LexA-Rad51 fusion protein includes, e.g., the coding sequence for the 1 to the 202 amino acid residues of the LexA protein. The linker separating the LexA and Rad51 proteins may be any linker known in the art, such as a polyglycine linker, as well as Glycine-Serine linkers. The Rad51 portion of the LexA-Rad51 fusion protein includes, e.g., the coding sequence for 210 to the 611 amino acid residues of the Rad51 protein. The ADH1 terminator is but one terminator that may be used in the fusion protein construct, other terminators include CYC1, GPD, ACT1, TEF1, RNR2, CYC1, TEF2, HXT7, YEF3, RPL3, or RPL4. The LexA binding domain may include one or more LexA binding domains. The LexA binding domain comprises a 16 bp tract of nucleotides; namely CTGTATATATATACAG.

FIG. 2B is a simplified graphic of enhancing homologous recombination—and thus increasing editing and possibly enhanced tyrosine production—using the LexA-Rad51 fusion protein in a yeast genome. In the graphic process 200 of FIG. 2B, a nucleic acid-guided nuclease binds to a target genomic sequence and creates a double strand break 203 in the target genomic sequence. The double strand break may be resolved in one of three ways. First, the double strand break may not be repaired and, if not repaired, the cell dies 205. Alternatively, the double strand break may be repaired by non-homologous end joining 207 leading to joining of the ends of a break without homology-directed repair, which is intrinsically mutagenic. Finally, the repair may be made by homologous repair 209 via a homologous sequence (e.g., donor DNA comprising the desired edit), leading to a desired sequence change. In the present disclosure, homologous repair is optimized by recruiting the editing plasmid comprising the donor DNA to the site of the double stranded break via a LexA-Rad51 fusion protein.

Following the arrow to homologous recombination in FIG. 2B, the editing complex is shown at 214; the editing plasmid is shown at 210; the target genomic sequence is shown at 220; the donor DNA (region of homology surrounding the desired edit) is shown at 212; the LexA-Rad51 fusion is shown generally at 218, with component LexA 224 shown bound to a LexA DNA binding domain on editing plasmid 210 and component Rad51 226 as a part of a Rad51 helical multimer 216 proximal to the cut site on target genomic sequence 220. Once homologous recombination has taken place, there should be a precise edit 222 on the target genomic sequence 220.

FIG. 2C is an exemplary editing vector map comprising a coding sequence for the LexA-Rad51 fusion protein, a CREATE cassette, and the coding sequence for the nuclease MAD7. Beginning at 11:55 o'clock, there is an pSNR52 promoter driving transcription of the gRNA, a penta-T motif, and a donor DNA sequence (e.g., one of the edits to the ARO4 gene or other gene of the shikimate pathway, knock outs to genes in the S. cerevisiae genome and synthetic terminators in the S. cerevisiae genome) followed by a SUP4 terminator; a pTEF promoter driving transcription of kanamycin resistance gene followed by a TEF terminator; an pADH1 promoter driving transcription of a LexA-linker-Rad51 fusion protein coding sequence followed by an ADH1 terminator; one or more LexA DNA binding sequences; another promoter driving transcription of an SV40 nuclear localization sequence and the MAD7 nuclease coding sequence followed by a CYC1 terminator; a promoter driving an ampicillin resistance gene (which is in a reverse orientation to the transcription of the other elements; a pUC origin of replication for propagation of the editing vector in bacteria; and a 2-μ origin of replication for propagation of the editing vector in yeast.

The nucleic acid mutations or edits described herein were generated using MAD7, along with a gRNA and donor DNA. A nucleic acid-guided nuclease such as MAD7 is complexed with an appropriate synthetic guide nucleic acid in a cell and can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid may be a single guide nucleic acid that includes both the crRNA and tracrRNA sequences.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In the methods to generate the mutant library for screening for tyrosine, the guide nucleic acids were provided as a sequence to be expressed from a plasmid or vector comprising both the guide sequence and the scaffold sequence as a single transcript under the control of an inducible promoter. The guide nucleic acids are engineered to target a desired target sequence by altering the guide sequence so that the guide sequence is complementary to a desired target sequence, thereby allowing hybridization between the guide sequence and the target sequence. In general, to generate an edit in the target sequence, the gRNA/nuclease complex binds to a target sequence as determined by the guide RNA, and the nuclease recognizes a proto spacer adjacent motif (PAM) sequence adjacent to the target sequence. The target sequences for the genome-wide mutagenesis here encompassed approximately 20,000 loci throughout the E. coli genome.

The guide nucleic acid may be and in the processes of generating the variants reported herein were part of an editing cassette that also encoded the donor nucleic acid. The target sequences are associated with a proto-spacer mutation (PAM), which is a short nucleotide sequence recognized by the gRNA/nuclease complex. The precise preferred PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence.

In certain embodiments, the genome editing of a cellular target sequence both introduces the desired DNA change to the cellular target sequence and removes, mutates, or renders inactive a proto-spacer mutation (PAM) region in the cellular target sequence. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease component of the nucleic acid-guided nuclease editing system, a polynucleotide sequence encoding the nucleic acid-guided nuclease can be codon optimized for expression in particular cell types, such as archaeal, prokaryotic or eukaryotic cells. The choice of nucleic acid-guided nuclease to be employed depends on many factors, such as what type of edit is to be made in the target sequence and whether an appropriate PAM is located close to the desired target sequence. Nucleases of use in the methods described herein include but are not limited to Cas 9, Cas 12/CpfI, MAD2, or MAD7 or other MADzymes. As with the guide nucleic acid, the nuclease is encoded by a DNA sequence on a vector (e.g., the engine vector—see FIG. 3A) and be under the control of an inducible promoter. In some embodiments—such as in the methods described herein—the inducible promoter may be separate from but the same as the inducible promoter controlling transcription of the guide nucleic acid; that is, a separate inducible promoter drives the transcription of the nuclease and guide nucleic acid sequences but the two inducible promoters may be the same type of inducible promoter (e.g., both are pL promoters). Alternatively, the inducible promoter controlling expression of the nuclease may be different from the inducible promoter controlling transcription of the guide nucleic acid; that is, e.g., the nuclease may be under the control of the pBAD inducible promoter, and the guide nucleic acid may be under the control of the pL inducible promoter.

Another component of the nucleic acid-guided nuclease system is the donor nucleic acid comprising homology to the cellular target sequence. In some embodiments, the donor nucleic acid is on the same polynucleotide (e.g., editing vector or editing cassette) as the guide nucleic acid. The donor nucleic acid is designed to serve as a template for homologous recombination with a cellular target sequence nicked or cleaved by the nucleic acid-guided nuclease as a part of the gRNA/nuclease complex. A donor nucleic acid polynucleotide may be of any suitable length, such as about or more than about 20, 25, 50, 75, 100, 150, 200, 500, or 1000 nucleotides in length. In certain preferred aspects, the donor nucleic acid can be provided as an oligonucleotide of between 20-300 nucleotides, more preferably between 50-250 nucleotides. The donor nucleic acid comprises a region that is complementary to a portion of the cellular target sequence (e.g., a homology arm). When optimally aligned, the donor nucleic acid overlaps with (is complementary to) the cellular target sequence by, e.g., about 20, 25, 30, 35, 40, 50, 60, 70, 80, 90 or more nucleotides. In many embodiments, the donor nucleic acid comprises two homology arms (regions complementary to the cellular target sequence) flanking the mutation or difference between the donor nucleic acid and the cellular target sequence. The donor nucleic acid comprises at least one mutation or alteration compared to the cellular target sequence, such as an insertion, deletion, modification, or any combination thereof compared to the cellular target sequence. Various types of edits were introduced herein, including site-directed mutagenesis, saturation mutagenesis, promoter swaps and ladders, knock-in and knock-out edits, SNP or short tandem repeat swaps, and start/stop codon exchanges.

In addition to the donor nucleic acid, an editing cassette may comprise one or more primer sites. The primer sites can be used to amplify the editing cassette by using oligonucleotide primers; for example, if the primer sites flank one or more of the other components of the editing cassette. In addition, the editing cassette may comprise a barcode. A barcode is a unique DNA sequence that corresponds to the donor DNA sequence such that the barcode can identify the edit made to the corresponding cellular target sequence. The barcode typically comprises four or more nucleotides. In some embodiments, the editing cassettes comprise a collection or library gRNAs and of donor nucleic acids representing, e.g., gene-wide or genome-wide libraries of gRNAs and donor nucleic acids. The library of editing cassettes is cloned into vector backbones where, e.g., each different donor nucleic acid is associated with a different barcode.

Variants of interest discovered by the methods herein during a first round of screening include those listed in Table 1 below:

TABLE 1
Variants

| | Gene | SGD | | NCBI- | | | |
|---|---|---|---|---|---|---|---|
| Seq ID No. | Name | Systematic ID | SGD ID | GeneID | Edit Type | Variant | FIOWT |
| SEQ ID No. 1 | ALY1 | YKR021W | S000001729 | 853891 | stop | I46*** | 1.800194574 |
| SEQ ID No. 2 | ARO1 | YDR127W | S000002534 | 851705 | AA swap | I144D | 1.154062558 |
| SEQ ID No. 3 | ARO1 | YDR127W | S000002534 | 851705 | AA swap | K249M | 1.184880381 |
| SEQ ID No. 4 | ARO1 | YDR127W | S000002534 | 851705 | AA swap | C298Q | 1.269929652 |
| SEQ ID No. 5 | ARO1 | YDR127W | S000002534 | 851705 | AA swap | M303Q | 1.148342529 |
| SEQ ID No. 6 | ARO4 | YBR249C | S000000453 | 852551 | AA swap | S4K | 1.325196041 |
| SEQ ID No. 7 | ARO4 | YBR249C | S000000453 | 852551 | AA swap | N10R | 1.12393312 |
| SEQ ID No. 8 | ARO4 | YBR249C | S000000453 | 852551 | AA swap | D22T | 2.140124686 |
| SEQ ID No. 9 | ARO4 | YBR249C | S000000453 | 852551 | AA swap | A32Y | 1.203838363 |
| SEQ ID No. 10 | ARO4 | YBR249C | S000000453 | 852551 | AA swap | P34G | 2.013585738 |
| SEQ ID No. 11 | ARO4 | YBR249C | S000000453 | 852551 | AA swap | T44R | 1.42741611 |
| SEQ ID No. 12 | ARO4 | YBR249C | S000000453 | 852551 | AA swap | P45V | 2.115872846 |
| SEQ ID No. 13 | ARO4 | YBR249C | S000000453 | 852551 | AA swap | L48C | 1.718167648 |
| SEQ ID No. 14 | ARO4 | YBR249C | S000000453 | 852551 | AA swap | A83V | 1.541135856 |
| SEQ ID No. 15 | ARO4 | YBR249C | S000000453 | 852551 | AA swap | N133Q | 1.676981331 |
| SEQ ID No. 16 | ARO4 | YBR249C | S000000453 | 852551 | AA swap | N135T | 2.115872846 |
| SEQ ID No. 17 | ARO4 | YBR249C | S000000453 | 852551 | AA swap | K136R | 1.14935202 |
| SEQ ID No. 18 | ARO4 | YBR249C | S000000453 | 852551 | AA swap | S140A | 1.951386417 |
| SEQ ID No. 19 | ARO4 | YBR249C | S000000453 | 852551 | AA swap | S140R | 1.917600091 |
| SEQ ID No. 20 | ARO4 | YBR249C | S000000453 | 852551 | AA swap | S173W | 1.686388665 |
| SEQ ID No. 21 | ARO4 | YBR249C | S000000453 | 852551 | AA swap | Q185A | 1.654101437 |
| SEQ ID No. 22 | ARO4 | YBR249C | S000000453 | 852551 | AA swap | L194Y | 1.910320212 |
| SEQ ID No. 23 | ARO4 | YBR249C | S000000453 | 852551 | AA swap | S195G | 2.45611319 |
| SEQ ID No. 24 | ARO4 | YBR249C | S000000453 | 852551 | AA swap | S195W | 2.388632117 |
| SEQ ID No. 25 | ARO4 | YBR249C | S000000453 | 852551 | AA swap | S195W | 2.377765966 |
| SEQ ID No. 26 | ARO4 | YBR249C | S000000453 | 852551 | AA swap | S195M | 2.136088709 |
| SEQ ID No. 27 | ARO4 | YBR249C | S000000453 | 852551 | AA swap | F196Q | 2.089768381 |
| SEQ ID No. 28 | ARO4 | YBR249C | S000000453 | 852551 | AA swap | G199V | 1.221070011 |
| SEQ ID No. 29 | ARO4 | YBR249C | S000000453 | 852551 | AA swap | G203I | 1.908943994 |
| SEQ ID No. 30 | ARO4 | YBR249C | S000000453 | 852551 | AA swap | G203A | 1.842099127 |
| SEQ ID No. 31 | ARO4 | YBR249C | S000000453 | 852551 | AA swap | T204C | 1.598758696 |
| SEQ ID No. 32 | ARO4 | YBR249C | S000000453 | 852551 | AA swap | A211S | 1.908943994 |
| SEQ ID No. 33 | ARO4 | YBR249C | S000000453 | 852551 | AA swap | C215E | 2.347777833 |
| SEQ ID No. 34 | ARO4 | YBR249C | S000000453 | 852551 | AA swap | H223Q | 1.221070011 |
| SEQ ID No. 35 | ARO4 | YBR249C | S000000453 | 852551 | AA swap | F224Q | 2.003196725 |
| SEQ ID No. 36 | ARO4 | YBR249C | S000000453 | 852551 | AA swap | M225A | 1.581939281 |
| SEQ ID No. 37 | ARO4 | YBR249C | S000000453 | 852551 | AA swap | V227P | 2.410513157 |
| SEQ ID No. 38 | ARO4 | YBR249C | S000000453 | 852551 | AA swap | K229I | 2.62563039 |
| SEQ ID No. 39 | ARO4 | YBR249C | S000000453 | 852551 | AA swap | E242M | 2.136088709 |
| SEQ ID No. 40 | ARO4 | YBR249C | S000000453 | 852551 | AA swap | C244T | 2.45297226 |
| SEQ ID No. 41 | ARO4 | YBR249C | S000000453 | 852551 | AA swap | F245H | 1.24009441 |
| SEQ ID No. 42 | ARO4 | YBR249C | S000000453 | 852551 | AA swap | I247Q | 1.292471632 |
| SEQ ID No. 43 | ARO4 | YBR249C | S000000453 | 852551 | AA swap | I247M | 1.434565907 |
| SEQ ID No. 44 | ARO4 | YBR249C | S000000453 | 852551 | AA swap | L248E | 1.543963533 |
| SEQ ID No. 45 | ARO4 | YBR249C | S000000453 | 852551 | AA swap | R249G | 2.315900983 |
| SEQ ID No. 46 | ARO4 | YBR249C | S000000453 | 852551 | AA swap | R249L | 1.357442285 |
| SEQ ID No. 47 | ARO4 | YBR249C | S000000453 | 852551 | AA swap | G250E | 1.772609757 |
| SEQ ID No. 48 | ARO4 | YBR249C | S000000453 | 852551 | AA swap | K253Q | 1.224474226 |
| SEQ ID No. 49 | ARO4 | YBR249C | S000000453 | 852551 | AA swap | K253L | 2.047771942 |
| SEQ ID No. 50 | ARO4 | YBR249C | S000000453 | 852551 | AA swap | K253P | 1.777900498 |
| SEQ ID No. 51 | ARO4 | YBR249C | S000000453 | 852551 | AA swap | N256Q | 1.811391257 |
| SEQ ID No. 52 | ARO4 | YBR249C | S000000453 | 852551 | AA swap | D258P | 2.282166577 |
| SEQ ID No. 53 | ARO4 | YBR249C | S000000453 | 852551 | AA swap | D258W | 2.027815994 |
| SEQ ID No. 53 | ARO4 | YBR249C | S000000453 | 852551 | AA swap | D258W | 2.027815994 |
| SEQ ID No. 54 | ARO4 | YBR249C | S000000453 | 852551 | AA swap | S281G | 1.440200181 |
| SEQ ID No. 55 | ARO4 | YBR249C | S000000453 | 852551 | AA swap | N284P | 2.115872846 |
| SEQ ID No. 56 | ARO4 | YBR249C | S000000453 | 852551 | AA swap | S285P | 1.674285482 |
| SEQ ID No. 57 | ARO4 | YBR249C | S000000453 | 852551 | AA swap | K294H | 1.404905046 |
| SEQ ID No. 58 | ARO4 | YBR249C | S000000453 | 852551 | AA swap | G306N | 1.249047018 |
| SEQ ID No. 59 | ARO4 | YBR249C | S000000453 | 852551 | AA swap | Q324I | 1.658506462 |
| SEQ ID No. 60 | ARO4 | YBR249C | S000000453 | 852551 | AA swap | T350L | 1.402900693 |
| SEQ ID No. 61 | ARO7 | YPR060C | S000006264 | 856173 | AA swap | V20C | 1.800285889 |
| SEQ ID No. 62 | ARO7 | YPR060C | S000006264 | 856173 | AA swap | Y43W | 1.104970477 |
| SEQ ID No. 63 | ARO7 | YPR060C | S000006264 | 856173 | AA swap | L67H | 1.242796462 |
| SEQ ID No. 64 | ARO7 | YPR060C | S000006264 | 856173 | AA swap | L179V | 1.105696316 |
| SEQ ID No. 65 | ARO7 | YPR060C | S000006264 | 856173 | AA swap | M189A | 1.267198849 |
| SEQ ID No. 66 | ARO7 | YPR060C | S000006264 | 856173 | AA swap | K190S | 2.292057144 |
| SEQ ID No. 67 | ARO7 | YPR060C | S000006264 | 856173 | AA swap | V211K | 2.660289022 |
| SEQ ID No. 68 | AVT3 | YKL146W | S000001629 | 853710 | terminator | Tsynth12 | 1.264571834 |
| SEQ ID No. 69 | CAF130 | YGR134W | S000003366 | 853035 | terminator | Tsynth12 | 1.499611886 |
| SEQ ID No. 70 | CSM4 | YPL200W | S000006121 | 855901 | terminator | Tsynth12 | 1.126351771 |
| SEQ ID No. 71 | FTR1 | YER145C | S000000947 | 856888 | stop | L32*** | 1.455448396 |
| SEQ ID No. 72 | FUM1 | YPL262W | S000006183 | 855866 | stop | A42*** | 2.391078494 |
| SEQ ID No. 73 | GCS1 | YDL226C | S000002385 | 851372 | stop | E48*** | 1.107838983 |
| SEQ ID No. 74 | GPI2 | YPL076W | S000005997 | 856029 | terminator | Tsynth12 | 1.232852203 |
| SEQ ID No. 75 | HIS2 | YFR025C | S000001921 | 850581 | stop | E38*** | 1.311660958 |
| SEQ ID No. 76 | IES1 | YFL013C | S000001881 | 850534 | stop | E132*** | 2.934699697 |
| SEQ ID No. 77 | IMP2′ | YIL154C | S000001416 | 854652 | terminator | Tsynth12 | 2.348729933 |
| SEQ ID No. 78 | KTI11 | YBL071W-A | S000007587 | 852207 | terminator | Tsynth12 | 1.353345474 |
| SEQ ID No. 79 | MET6 | YER091C | S000000893 | 856825 | stop | K48*** | 4.120350113 |
| SEQ ID No. 80 | MRPL20 | YKR085C | S000001793 | 853960 | terminator | Tsynth12 | 2.010141477 |
| SEQ ID No. 81 | MTQ2 | YDR140W | S000002547 | 851718 | terminator | Tsynth12 | 1.20203406 |
| SEQ ID No. 82 | RPE1 | YJL121C | S000003657 | 853322 | stop | G49*** | 2.170464817 |
| SEQ ID No. 82 | RPE1 | YJL121C | S000003657 | 853322 | stop | G49*** | 1.826617747 |
| SEQ ID No. 83 | SDH1 | YKL148C | S000001631 | 853709 | terminator | Tsynth12 | 1.15168315 |
| SEQ ID No. 84 | SHQ1 | YIL104C | S000001366 | 854702 | terminator | Tsynth12 | 1.150376868 |
| SEQ ID No. 85 | TAZ1 | YPR140W | S000006344 | 856262 | terminator | Tsynth12 | 2.111959932 |
| SEQ ID No. 86 | YCR051W | YCR051W | S000000647 | 850418 | stop | N58*** | 1.770476171 |
| SEQ ID No. 87 | YIL001W | YIL001W | S000001263 | 854816 | terminator | Tsynth12 | 1.286863094 |
| SEQ ID No. 88 | YNL190W | YNL190W | S000005134 | 855531 | terminator | Tsynth12 | 1.729253911 |
| SEQ ID No. 89 | ZIP2 | YGL249W | S000003218 | 852643 | stop | L47*** | 1.78250108 |
| SEQ ID No. 90 | AAT2 | YLR027C | S000004017 | 850714 | stop | K85*** | 2.4556536 |
| SEQ ID No. 91 | ARO4 | YBR249C | S000000453 | 852551 | AA swap | V212Y | 2.1887702 |
| SEQ ID No. 92 | ARO4 | YBR249C | S000000453 | 852551 | AA swap | D80K | 1.902787264 |
| SEQ ID No. 93 | ARO4 | YBR249C | S000000453 | 852551 | AA swap | D213R | 1.897929941 |
| SEQ ID No. 94 | ARO4 | YBR249C | S000000453 | 852551 | AA swap | N256N | 1.6150426 |
| SEQ ID No. 95 | ARO4 | YBR249C | S000000453 | 852551 | AA swap | S77D | 1.61370638 |
| SEQ ID No. 96 | ARO4 | YBR249C | S000000453 | 852551 | AA swap | V198I | 1.577256222 |
| SEQ ID No. 97 | ARO4 | YBR249C | S000000453 | 852551 | AA swap | I247F | 1.571632801 |
| SEQ ID No. 98 | ARO4 | YBR249C | S000000453 | 852551 | AA swap | H243H | 1.377523253 |
| SEQ ID No. 99 | RPN1 | YHR027C | S000001069 | 856422 | stop | R55*** | 1.418971445 |

In Table 1, “Gene Name” is the common abbreviated gene name, “SGD Systematic Name” is the Yeast Genome Database gene name (S. cerevisiae), “SGD ID” is the unique identifier in the SGD Yeast Genome Database, “NCBI-GeneID” is the NCBI accession number, “Edit Type” is one of an amino acid swap, stop codon, or terminator edit, “Variant” is the change to the wildtype sequence, “FIOWT” is fold increase over wild type; and *** is for hits from the genome-wide knock out library where a triple-stop was inserted at a given position in the locus.

### EXAMPLES

Mutagenesis libraries specifically targeting the genes the tyrosine/shikimate pathway were designed for saturation mutagenesis. Additionally, to more deeply explore the rest of the S. cerevisiae genome for new targets involved in tyrosine biosynthesis, libraries were designed to target all annotated loci with either premature stop codons (for a knock-out phenotype) or with an insertion of a set of five synthetic terminator variants (for expression modulation phenotypes). All libraries were screened at shallow sampling for tyrosine production via mass spec as described below.

### Example I

### Editing Cassette Preparation

5 nM oligonucleotides synthesized on a chip were amplified using Q5 polymerase in 50 μL volumes. The PCR conditions were 95° C. for 1 minute; 8 rounds of 95° C. for 30 seconds/60° C. for 30 seconds/72° C. for 2.5 minutes; with a final hold at 72° C. for 5 minutes. Following amplification, the PCR products were subjected to SPRI cleanup, where 30 μL SPRI mix was added to the 50 μL PCR reactions and incubated for 2 minutes. The tubes were subjected to a magnetic field for 2 minutes, the liquid was removed, and the beads were washed 2× with 80% ethanol, allowing 1 minute between washes. After the final wash, the beads were allowed to dry for 2 minutes, 50 μL 0.5× TE pH 8.0 was added to the tubes, and the beads were vortexed to mix. The slurry was incubated at room temperature for 2 minutes, then subjected to the magnetic field for 2 minutes. The eluate was removed and the DNA quantified.

Following quantification, a second amplification procedure was carried out using a dilution of the eluate from the SPRI cleanup. PCR was performed under the following conditions: 95° C. for 1 minute; 18 rounds of 95° C. for 30 seconds/72° C. for 2.5 minutes; with a final hold at 72° C. for 5 minutes. Amplicons were checked on a 2% agarose gel and pools with the cleanest output(s) were identified. Amplification products appearing to have heterodimers or chimeras were not used.

### Example II

### Backbone Preparation

Purified backbone vector was linearized by restriction enzyme digest with StuI. Up to 20 μg of purified backbone vector was in a 100 μL total volume in StuI-supplied buffer. Digestion was carried out at 30° C. for 16 hrs. Linear backbone was dialyzed to remove salt on 0.025 μm MCE membrane for ˜60 min on nuclease-free water. Linear backbone concentration was measured using dye/fluorometer-based quantification.

### Example III

### Preparation of Competent Cells

The afternoon before transformation was to occur, 10 mL of YPAD was added to S. cerevisiae cells, and the culture was shaken at 250 rpm at 30° C. overnight. The next day, approximately 2 mL of the overnight culture was added to 100 mL of fresh YPAD in a 250-mL baffled flask and grown until the OD600 reading reached 0.3+/−0.05. The culture was then placed in a 30° C. incubator shaking at 250 rpm and allowed to grow for 4-5 hours, with the OD checked every hour. When the culture reached ˜1.5 OD600, two 50 mL aliquots of the culture were poured into two 50-mL conical vials and centrifuged at 4300 rpm for 2 minutes at room temperature. The supernatant was removed from the 50 mL conical tubes, avoiding disturbing the cell pellet. 25 mL of lithium acetate/DTT solution was added to each conical tube and the pellet was gently resuspended using an inoculating loop, needle, or long toothpick.

Following resuspension, both cell suspensions were transferred to a 250-mL flask and placed in the shaker to shake at 30° C. and 200 rpm for 30 minutes. After incubation was complete, the suspension was transferred to one 50-mL conical tube and centrifuged at 4300 RPM for 3 minutes. The supernatant was then discarded. From this point on, cold liquids were used and kept on ice until electroporation was complete. 50 mL of 1 M sorbitol was added to the cells and the pellet was resuspended. The cells were centrifuged at 4300 rpm for 3 minutes at 4° C., and the supernatant was discarded. The centrifugation and resuspension steps were repeated for a total of three washes. 50 μL of 1 M sorbitol was then added to one pellet, the cells were resuspended, then this aliquot of cells was transferred to the other tube and the second pellet was resuspended. The approximate volume of the cell suspension was measured, then brought to a 1 mL volume with cold 1 M sorbitol. The cell/sorbitol mixture and transferred into a 2-mm cuvette. Impedance measurement of the cells was measured in the cuvette. At this point the KW must be ≥20. If this is not the case the cells should be washed in cold sorbitol two to three additional times.

Transformation was then performed using 500 ng of linear backbone along with 50 ng editing cassettes with the competent S. cerevisiae cells. 2 mm electroporation cuvettes were placed on ice and the plasmid/cassette mix was added to each corresponding cuvette. 100 μL of electrocompetent cells were added to each cuvette and the linear backbone and cassettes. Each sample was electroporated using the following conditions on a NEPAGENE electroporator: Poring pulse: 1800V, 5.0 second pulse length, 50.0 msec pulse interval, 1 pulse; Transfer pulse: 100 V, 50.0 msec pulse length, 50.0 msec pulse interval, with 3 pulses. Once the transformation process is complete, 900 μL of room temperature YPAD Sorbitol media was added to each cuvette. The cells were then transferred and suspended in a 15 mL tube and incubated shaking at 250 RPM at 30° C. for 3 hours. 9 mL of YPAD and 10 μL of Hygromycin B 1000× stock was added to the 15 mL tube.

### Example VI

### Screening of Edited Libraries for Tyrosine Production

Library stocks were diluted and plated onto 245×245 mm YPD agar plates (Teknova) containing 250 μg/mL Hygromycin (Teknova) using sterile glass beads. Libraries were diluted an appropriate amount to yield ˜1500-2000 colonies on the plates. Plates were incubated 36-48 h at 30° C. and then stored at 4° C. until use. Colonies were picked using a QPix™ 420 (Molecular Devices) and deposited into sterile 1.2 mL square 96-well plates (Thomas Scientific) containing 300 μL of growth medium (Synthetic Complete (SC) medium (-Tyr, -Phe) containing 250 μg/mL Hygromycin (Sigma)). SC (-Tyr, -Phe) medium was prepared by adding 6.71 g/L YNB+nitrogen (AmSO4) (Sunrise Science Products), 0.69 g/L CSM (-Tyr, -Phe) (Sunrise Science Products) and 20 g/L glucose (Tenova) to water. Plates were sealed (AirPore sheets (Qiagen)) and incubated for 36-48 h in a shaker incubator (Climo-Shaker ISF1-X (Kuhner), 30° C., 85% humidity, 250 rpm). Plate cultures were then diluted 20-fold (15 μL culture into 285 μL medium) into new 96-well plates containing fresh SC (-Tyr, -Phe) medium. Production plates were incubated for 24 h in a shaker incubator (Climo-Shaker ISF1-X (Kuhner), 30° C., 85% humidity, 250 rpm).

Production plates were centrifuged (Centrifuge 5920R, Eppendorf) at 3,000 g for 10 min to pellet cells. The supernatants from production plates were diluted 500-fold into water (10 μL of supernatant with 490 μL) of water in 1.2 mL square 96-well plates. Samples were thoroughly mixed and then diluted a subsequent 10-fold further into acetonitrile (LC/MS grade, Fisher), (20 μL sample with 180 μL of acetonitrile) into a 96-well Plate (polypropylene, 335 μL/well, Conical Bottom (Thomas Scientific). Plates were heat sealed and thoroughly mixed. Tyrosine concentrations were determined using a RapidFire high-throughput mass spectrometry system (Agilent) coupled to a 6470 Triple Quad mass spectrometer (Agilent). The RapidFire conditions were as follows: Pump 1: 95% acetonitrile (LC/MS grade, Fisher), 5% water (LC/MS grade, Fisher), 0.1% formic acid (Sigma) 1.5 mL/min, Pump 2: 100% water, 1.25 mL/min, Pump 3: 5% acetonitrile, 95% water, 1.25 mL/min. RapidFire method: Aspirate: 600 ms, Load/wash: 3000 ms, Extra wash: 0 ms, Elute: 3000 ms, Re-equilibrate: 500 ms. 10 μL injection loop.

Tyrosine concentrations were determined using a RapidFire high-throughput mass spectrometry system (Agilent) coupled to a 6470 Triple Quad mass spectrometer (Agilent). The RapidFire conditions were as follows: Pump 1: 95% acetonitrile (LC/MS grade, Fisher), 5% water (LC/MS grade, Fisher), 0.1% formic acid (Sigma) 1.5 mL/min, Pump 2: 100% water, 1.25 mL/min, Pump 3: 5% acetonitrile, 95% water, 1.25 mL/min. RapidFire method: Aspirate: 600 ms, Load/wash: 3000 ms, Extra wash: 0 ms, Elute: 3000 ms, Re-equilibrate: 500 ms. 10 μL injection loop.

### Example VI

### Mass Spectrometry Conditions for Tyrosine Detection

Precursor ion: 182.1 m/z, Product ion (quantifying): 136.0 m/z, Dwell: 50, Fragmentor: 72, Collision energy: 12, Cell accelerator voltage: 4, Polarity: positive

Precursor ion: 182.1 m/z, Product ion (qualifying): 91.0 m/z, Dwell: 50, Fragmentor: 72, Collision energy: 36, Cell accelerator voltage: 4, Polarity: positive

Source conditions: Gas Temp: 300° C., Gas Flow: 10 L/min, Nebulizer: 45 psi, Sheath gas temp: 350° C., Sheath gas flow: 11 L/min, Capillary voltage: 3500V (positive), Nozzle voltage: 500V (positive)

Data was analyzed using MassHunter Quantitative Analysis software (Agilent) with a standard curve of Tyrosine used for quantitation of Tyrosine in the samples. Each 96-well plate of samples contained 4 replicates of the wildtype strain to calculate the relative Tyrosine of samples compared to the base strain used for editing. Hits from the primary screen were re-tested in quadruplicate using a similar protocol as described above.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, [symbol] 6.

1. A composition of matter comprising a variant of the 3-Deoxy-D-arabino-heptulosonate-7-phosphate (DAHP) synthase (ARO4) gene selected from SEQ ID No. 23, SEQ ID No. 24, SEQ ID No. 25, SEQ ID No. 26, SEQ ID No. 33, SEQ ID No. 37, SEQ ID No. 38, SEQ ID No. 40, SEQ ID No. 45 or SEQ ID No. 52.

2. The composition of matter of claim 1 comprising a variant of the 3-Deoxy-D-arabino-heptulosonate-7-phosphate (DAHP) synthase (ARO4) gene, wherein the variant is SEQ ID No. 23.

3. The composition of matter of claim 1 comprising a variant of the 3-Deoxy-D-arabino-heptulosonate-7-phosphate (DAHP) synthase (ARO4) gene, wherein the variant is SEQ ID No. 24.

4. The composition of matter of claim 1 comprising a variant of the 3-Deoxy-D-arabino-heptulosonate-7-phosphate (DAHP) synthase (ARO4) gene, wherein the variant is SEQ ID No. 25.

5. The composition of matter of claim 1 comprising a variant of the 3-Deoxy-D-arabino-heptulosonate-7-phosphate (DAHP) synthase (ARO4) gene, wherein the variant is SEQ ID No. 26.

6. The composition of matter of claim 1 comprising a variant of the 3-Deoxy-D-arabino-heptulosonate-7-phosphate (DAHP) synthase (ARO4) gene, wherein the variant is SEQ ID No. 33.

7. The composition of matter of claim 1 comprising a variant of the 3-Deoxy-D-arabino-heptulosonate-7-phosphate (DAHP) synthase (ARO4) gene, wherein the variant is SEQ ID No. 37.

8. The composition of matter of claim 1 comprising a variant of the 3-Deoxy-D-arabino-heptulosonate-7-phosphate (DAHP) synthase (ARO4) gene, wherein the variant is SEQ ID No. 38.

9. The composition of matter of claim 1 comprising a variant of the 3-Deoxy-D-arabino-heptulosonate-7-phosphate (DAHP) synthase (ARO4) gene, wherein the variant is SEQ ID No. 40.

10. The composition of matter of claim 1 comprising a variant of the 3-Deoxy-D-arabino-heptulosonate-7-phosphate (DAHP) synthase (ARO4) gene, wherein the variant is SEQ ID No. 45.

11. The composition of matter of claim 1 comprising a variant of the 3-Deoxy-D-arabino-heptulosonate-7-phosphate (DAHP) synthase (ARO4) gene, wherein the variant is SEQ ID No. 52.


SEQUENCE LISTING (99 sequences)

SEQ ID NO: 1 - protein, 912 aa, Saccharomyces cerevisiae
MLQFNTENDTVAPVFPMEQDINAAPDAVPLVQTTTLQVFVKLAEPLKGFETNGLSEIAPSILRGSLIVRVLKPNKLKSISITFKGISRTEWPEGIPPKREEFSDVETVVNHTWPFYQADDGMNSFTLEHHSSNNSSNRPSMSDEDYLLEKSGASVYIPPTAEPPKDNSNLSLDAYERNSLSSDNLSNKPVSSDVSHDDSKLLAIQKTPLPSSSRRGSVPANFHGNSLSPHTFISDLFTKTFSNSGATPSPEQEDNYLTPSKDSKEVFIFRPGDYIYTFEQPISQSYPESIKANFGSVEYKLSIDIERFGAFKSTIHTQLPIKVVRLPSDGSVEETEAIAISKDWKDLLHYDVVIFSKEIVLNAFLPIDFHFAPLDKVTLHRIRIYLTESMEYTCNSNGNHEKARRLEPTKKFLLAEHNGPKLPHIPAGSNPLKAKNRGNILLDEKSGDLVNKDFQFEVFVPSKFTNSIRLHPDTNYDKIKAHHWIKICLRLSKKYGDNRKHFEISIDSPIHILNQLCSHANTLLPSYESHFQYCDEDGNFAPAADQQNYASHHDSNIFFPKEVLSSPVLSPNVQKMNIRIPSDLPVVRNRAESVKKSKSDNTSKKNDQSSNVFASKQLVANIYKPNQIPRELTSPQALPLSPITSPILNYQPLSNSPPPDFDFDLAKRGAADSHAIPVDPPSYFDVLKADGIELPYYDTSSSKIPELKLNKSRETLASIEEDSFNGWSQIDDLSDEDDNDGDIASGFNFKLSTSAPSENVNSHTPILQSLNMSLDGRKKNRASLHATSVLPSTIRQNNQHFNDINQMLGSSDEDAFPKSQSLNFNKKLPILKINDNVIQSNSNSNNRVDNPEDTVDSSVDITAFYDPRMSSDSKFDWEVSKNHVDPAAYSVNVASENRVLDDFKKAFREKRK

SEQ ID NO: 2 - protein, 1588 aa, Saccharomyces cerevisiae
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSDGGKTAIDTPLGKNFIGAFWQPKFVLVDIKWLETLAKREFINGMAEVIKTACIWNADEFTRLESNASLFLNVVNGAKNVKVINQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSSDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTPSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVATPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTKKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKIIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKAPFKAIFDAVTKE

SEQ ID NO: 3 - protein, 1588 aa, Saccharomyces cerevisiae
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVINQLTNEIDEISNTDIEAMLDHTYMLVLESIKVKAEVVSSDERESSLRNLLNFGHSIGHAYEAILTPQALHGECVSIGMVKEAELSRYFGILSPTQVARLSKILVAYGLPVSSDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTPSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVATPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTKKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKIIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKAPFKAIFDAVTKE

SEQ ID NO: 4 - protein, 1588 aa, Saccharomyces cerevisiae
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVINQLTNEIDEISNTDIEAMLDHTYKLVLESIKVKAEVVSSDERESSLRNLLNFGHSIGHAYEAILTPQALHGEQVSIGMVKEAELSRYFGILSPTQVARLSKILVAYGLPVSSDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTPSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVATPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTKKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKIIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKAPFKAIFDAVTKE

SEQ ID NO: 5 - protein, 1588 aa, Saccharomyces cerevisiae
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVINQLTNEIDEISNTDIEAMLDHTYKLVLESIKVKAEVVSSDERESSLRNLLNFGHSIGHAYEAILTPQALHGECVSIGQVKEAELSRYFGILSPTQVARLSKILVAYGLPVSSDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTPSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVATPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTKKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKIIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKAPFKAIFDAVTKE

SEQ ID NO: 6 - protein, 370 aa, Saccharomyces cerevisiae
MSEKPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 7 - protein, 370 aa, Saccharomyces cerevisiae
MSESPMFAARGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 8 - protein, 370 aa, Saccharomyces cerevisiae
MSESPMFAANGMPKVNQGAEETVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 9 - protein, 370 aa, Saccharomyces cerevisiae
MSESPMFAANGMPKVNQGAEEDVRILGYDPLYSPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 10 - protein, 370 aa, Saccharomyces cerevisiae
MSESPMFAANGMPKVNQGAEEDVRILGYDPLASGALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 11 - protein, 370 aa, Saccharomyces cerevisiae
MSESPMFAANGMPKVNQGAEEDVRILGYDPLASPALLQVQIPARPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 12 - protein, 370 aa, Saccharomyces cerevisiae
MSESPMFAANGMPKVNQGAEEDVRILGYDPLASPALLQVQIPATVTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 13 - protein, 370 aa, Saccharomyces cerevisiae
MSESPMFAANGMPKVNQGAEEDVRILGYDPLASPALLQVQIPATPTSCETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 14 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEVAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 15 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFQINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 16 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNITKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 17 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINRGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 18 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQAARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 19 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQRARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 20 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVWFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 21 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESALHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 22 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGYSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 23 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLGFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 24 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLWFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 25 - protein, 370 aa, Saccharomyces cerevisiae; identical to SEQ ID NO: 24

SEQ ID NO: 26 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLMFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 27 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSQPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 28 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVVFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 29 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNITDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 30 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNATDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 31 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGCDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 32 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVSVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 33 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDAEQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 34 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHQFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 35 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHQMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 36 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFAGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 37 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGPTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 38 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTIHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 39 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNMHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 40 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHTFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 41 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCHVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 42 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVQLRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 43 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVMLRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 44 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVIERGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 45 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILGGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 46 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILLGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 47 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILREGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 48 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKQGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 49 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKLGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 50 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKPGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 51 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTQYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 52 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYPAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 53 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYWAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 54 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYGHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 55 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGPSNKDFRNQPKVNDVVCEQIANGENAITGVMIESNINEGNQGIPAEGKAGLKYGVSITDACIGWETTEDVLRKLAAAVRQRREVNKK

SEQ ID NO: 56 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNPNKDFRNQPKVNDVVCEQIANGENAITGVMIESNINEGNQGIPAEGKAGLKYGVSITDACIGWETTEDVLRKLAAAVRQRREVNKK

SEQ ID NO: 57 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPHVNDVVCEQIANGENAITGVMIESNINEGNQGIPAEGKAGLKYGVSITDACIGWETTEDVLRKLAAAVRQRREVNKK

SEQ ID NO: 58 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANNENAITGVMIESNINEGNQGIPAEGKAGLKYGVSITDACIGWETTEDVLRKLAAAVRQRREVNKK

SEQ ID NO: 59 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNIGIPAEGKAGLKYGVSITDACIGWETTEDVLRKLAAAVRQRREVNKK

SEQ ID NO: 60 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETLEDVLRKLAAAVRQRREVNKK

SEQ ID NO: 61 - protein, 256 aa, Saccharomyces cerevisiae
MDFTKPETVLNLQNIRDELCRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMKNITNSAVEEKILERLTKKAEVYGVDPTNESGERRITPEYLVKIYKEIVIPITKEVEVEYLLRRLEE

SEQ ID NO: 62 - protein, 256 aa, Saccharomyces cerevisiae
MDFTKPETVLNLQNIRDELVRMEDSIIFKFIERSHFATCPSVWEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMKNITNSAVEEKILERLTKKAEVYGVDPTNESGERRITPEYLVKIYKEIVIPITKEVEVEYLLRRLEE

SEQ ID NO: 63 - protein, 256 aa, Saccharomyces cerevisiae
MDFTKPETVLNLQNIRDELVRMEDSIIFKFIERSHFATCPSVYEANHPGLEIPNFKGSFLDWALSNHEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMKNITNSAVEEKILERLTKKAEVYGVDPTNESGERRITPEYLVKIYKEIVIPITKEVEVEYLLRRLEE

SEQ ID NO: 64 - protein, 256 aa, Saccharomyces cerevisiae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKVIKSKDVEGIMKNITNSAVEEKILERLTKKAEVYGVDPTNESGERRITPEYLVKIYKEIVIPITKEVEVEYLLRRLEE

SEQ ID NO: 65 - protein, 256 aa, Saccharomyces cerevisiae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIAKNITNSAVEEKILERLTKKAEVYGVDPTNESGERRITPEYLVKIYKEIVIPITKEVEVEYLLRRLEE

SEQ ID NO: 66 - protein, 256 aa, Saccharomyces cerevisiae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMSNITNSAVEEKILERLTKKAEVYGVDPTNESGERRITPEYLVKIYKEIVIPITKEVEVEYLLRRLEE

SEQ ID NO: 67 - protein, 256 aa, Saccharomyces cerevisiae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMKNITNSAVEEKILERLTKKAEKYGVDPTNESGERRITPEYLVKIYKEIVIPITKEVEVEYLLRRLEE

SEQ ID NO: 68 - DNA, 2268 bp, Saccharomyces cerevisiae
atgaatggaaaagaggtttcaagtggctctggaagaacgcagagcaacaacaacaaaaaaaataacaacggaggcagtacaggaatctcgcatgcgtctggctcacctttaacggacggcaacggtggcaatagcaacggtaatagtagaagtagaagcagaagtagaaaatctagtggaactacaggtggtttgctaaaaaagccgccgctgcttgtgaacaatgaagcggtgcatgctagcgtacccgacgcgtcgcacacttcctgtaacaatggcacgcttgaagtgagcataaataaccctgagccacatgtggtagatgccgtggcacgccacttgataagaaacccgagtaacagcctacaattgcaaggcggagacattacaagagatctgtataagtggacaaacgaccatccttcatcaccatcgcagtatcagtacccaagtcaacctgcactttccacctcaataccttcacaagcgccctcattctcgaatcgtaaaaggtctatgagcttttctgccgcttctatagcatcttcctctcaccttaacaacaactcggaggcgaatgggaatcctctagcggcaataggcctggcgcccgcaccgatgacgcacgaagaaatcagggctcctggtggattcagaagatcttttataatacagaagcgcaggaaacataacgttgatgcgcccataccgaattttttcaccaggaactttattgaatttttgaccttgtatggccacttcgctggtgaagatttatcggaagaagaggaggaggaggaagaaactgaagaggagccagaggaggaagctttggaaacggaaagcacccagctagtttctcgtgaacacgggcgccatcctcacaaatcatctacggtaaaggcggtgctgctcttgttaaagtcatttgttgggacaggtgtgcttttcctaccaaaggctttccataacggtggttggggatttagcgccctttgtctactctcgtgtgccctcatttcttatggatgtttcgtatcattgattaccaccaaggacaaagtaggcgtagatgggtacggtgacatgggtcgtatactatatgggcccaaaatgaaatttgccattctttcgtctatcgccttgtcacaaatcgggttttctgctgcatacactgttttcactgcaactaatttgcaggtcttttctgaaaatttcttccatttgaaaccgggcagcatcagtctagccacctatatctttgcacaagtactcatctttgtcccactatctttgacaagaaacatagccaagttaagtgggaccgcgttaatagcagatctatttattctactgggattggtttatgtttacgtttactccatttattatattgctgttaatggtgtcgcatccgatacaatgcttatgttcaataaagcggactggtcgttgttcatcggtaccgccatattcacctttgaaggtataggcctacttatccccattcaggagtcaatgaaacacccgaagcatttccggccatcgttgtccgcggtgatgtgtatcgtggcagtgattttcatatcatgtggtctcttatgctatgccgcttttggatctgacgtgaaaactgttgttttattgaattttccgcaggacacttcctatactctcactgttcaattactgtatgcattagccattttgttatccacgccgttgcagctatttcccgcgatacgtattttggaaaactggacttttccctcaaatgcatctggtaaatacaacccgaaagtcaaatggttgaaaaactatttccgttgcgctattgtcgtcctgacatctatcctcgcttgggtaggcgccaacgatttggacaaatttgtatcactggtaggctccttcgcgtgtatcccattgatctacatatacccgccattgttgcactataaggcgtccatcttatctggaacctcgagagccaggcttcttttagatttaatcgtcatagtatttggagttgctgtcatggcctacacgtcatggcagacgattaaaatgtggagccagtaatatataggtgcaggcaaataaagagtatcatctttcaaaatatatatatttatatatgactcgttatctatatgcatctggaacacacataccttgttttaattatgattagcataaatgtactatccttgtcaatttggtatggctgagtttacccggacgagaggaaaaggtatagtgaataggatt

SEQ ID NO: 69 - DNA, 3558 bp, Saccharomyces cerevisiae
atgacgaaaaagaaagcagctactaattatgctgaaaggcaaaatttagccagtgaagattcttcaggtgattcggttcattttaaggactttattcccttacaagagttactgaaagataaaaattatgttccatcggtggaaaatttggagaagatcttatataatgaaaccatgtttaatgatcagaaaatatgctctaacctattgcttgaggctctcataattacgttgttcacaacaatttcagggaagtcagctttacgattaattcagacttcttctttgaaggaaagaaaatcctgggcccagtcctttgaaaataacagttctagctatgcttccatagtactgagttggaaagataatgatatattactcctgaaattcttaaggtttttattagcaaataaaacggcccctcttcaaattaatcggtacaatcttccagaatacaaactcccgttgagctttttaattgtttcaaaaattactattccgtccatattactcaatgaaacgtataatttgttaaaggactatttgtattcgataactggccgtatagagagtttgataagctgcagctccacatttgataagccagcacttgtcgttagaaaaattttaaaagattacaatagaatgatagaatgccgaaatttttatttctggtattcctttaatgctgagaacagagtaaatcttacttttagtgataacattagcctgttaatggaaaatgatgaaggcaatgctggaagtggccttgacgatagccgttttgatcatcaaaaacaaccgagagaggcgataatgggccgaacaataaatgaccaggaacaaatatattcattcgaactgaaccaagatggaacgcttgaaatacccaacgttatggagcattctttactaagacacgagcttctttttaaaatattaaatctcactacggtattgacgccattattggaactacaattttctacgctatgtggcttagtagatccattaatgcagcctactcccaatgataagcatattatatcgatagactttctttttcagctttttttgggattaatgtctcagtcgatcaagacttctcaagaacacaacgaccattatgactggaaattttatatgtgttttaacatgcaaaaaattattgatgctaccatgttgaggctcaactgctttgattttgatatattaaactcggtaaacaatacagataatgcagttcattggaagacgcaacttcacagatggctaccgcatggcctgaatacacaagacttggaattactctatatgattgatatactggcggtatataccatctacaagttgtatgaaaaaatacctatacaactaaacccatttttattttcattaatatccttatggaaaaacctctcctgtgttatacttttagccttagaaattgatagaattgaagaggagaatggcacttacgaaacgccgcttatggttcgcgccacaattcgtggagccgcagcattgaggtctgtaatagctaccgttctcaatggattagtgaaaaataatgatcatgatttcaagcacgagtctttaaacacattcatgtcgccctatggaagaaaattgtgtcatggtgctttatatgccgatttaaggtcacatactgcatctctacttgcgcttggagcaagcatcgaagatgtaacagatttatttgccgatttacagtcaggtgatagatttgatgaggatattagatatatgtttgattatgaatgtgaagattatgatgaatctttctctgaaagcgatcatggaggattggatgaaagcgttgttaacccaacagaaaaaatagcatctggtagtaataatgttttctttcggagacggtgcaattgcatcttcaatgatgataagttggtggcagaggatggtgcaaatgaagcctttgggtcaacaaatagcgaaaacgttgaaggcgcaatgcataataataggaatgcggtacataacgcgactacagccacttctgatcacgtcgttacgtcacctaacccactttccgttagatcaagaagtacttttgaatttgactatagtggggaagattggagggacgttcccagagatttcaatatgtactattcgccatcttattcattcatccatgagcctaagttagatgtcatatttagtttgactctacgtggcgcaacagaaaagttgaataaggaggaatccatattgttagtgcgttcagttgcatcgtgtgtaaggaacgagcaggaccaaatgattttagcagacctggagtctaatttttccgcaagtataaacggcgatgtagaaggtgaaggaaataccaaaatgtccaagatagataatgaagatctcagaagaaccacgccggatgatatttatgaaatatggtctgaagaatcagcatttgaaaggatgctaaacgtgaatcacgacgttgcgtggagattaatggatgaaatgttaatgtgcaccggttataggagaatattgatatggttccttacgcatttggagttaaaacattcgttgatatattacgtttttgaactaataatggggttacgtggaaaaccgttttccggagaagccagtgatcaagataaaaaggatgatatgatatatgagatcttgaagaaaaagcagaagaatgaagatgcttcaggacttcccttttcaaggcaaggacctatcgtattatctgatattgagactaaaatgttactgcaggagttttttatgaatgctgctatttttttgtcatcaaaaaataacgaagaagagaatgaagacggcgagaaaatctccttgtactcccttggtttggttaggttgatctgttacatggtgcagacactcatcgcgaacgacaagtttttttttaccaaatcagaatgcacttttgagcttcaaactttactgatgacatggattggcatccttcctgaagccaaagatttattctttaagatcaagacaaggctcgccatggaagaagaggatagtgccgatactatgcagcatgagggcaggaagaactctgacatagagaagaagctaaacgctaagccagcttctgaactgaatctgaaattgttaaacctgtttccctcaaagcctgcaaacaaagacgatagttcccctattaacacgttgcgtagttttatcgctgattactccttcgacacccaggtgaaccctccaggaagaagggtggtgttctacgatggtaagattttgccattgcccaaagccgataagcctatcccacttcatgaatatataacactcgcagagctcgatgttggagacagtgagtgatatataggtgcaggcaaataaagagtatcatctttcaaatgacaattagccatgtaataacatcttgaggcagttaaatattcgttaccctgcaggtggcaaaaaatttatagaataaaagcataaaaagatggatatctatgtaataaggaaacattggcagagcgaagagaacagactgctttctat

SEQ ID NO: 70 - DNA, 660 bp, Saccharomyces cerevisiae
atgatggatggttccataacaaggaaggtaacatcaacgttgtccaaccaattggcgacctggaaatggaaactgcagttatcgttattggaaagaaaattagcgacaataaacaatgattattttctcctgcaatgggaactcctttttataactaatgaagtgatgaaatggaaagaaatgattgcctttttggaaagccagttattttgcacaacgcaaaatttcgtggcacaagagacacatgacagggaaacctttcaaagcctggtagatgattataacaaacagttgagcgaaaataacctgattatttctgtattgaaaagtagaccacagctatcatcatttcctatatatttgagcgatgaggtgtgttcacaccttaaattcgtcatcgcggaactaaattcattgataatagttttttttatttctttagtatttttgtgggtatcgatagaagtttaatatataggtgcaggcaaataaagagtatcatctttcaaataaaaaaaatacagtcttaggattattctctatttatcctcacgccaaactttgtatacaattacaaaagattttgaatatactagtcaaagtactatctttgttgtcattgccactgttattataattctgagcttgaccttgaggctg

SEQ ID NO: 71 - protein, 31 aa, Saccharomyces cerevisiae
MPNKVFNVAVFFVVFRECLEAVIVISVLLSF

SEQ ID NO: 72 - protein, 41 aa, Saccharomyces cerevisiae
MLRFTNCSCKTFVKSSYKLNIRRMNSSFRTETDAFGEIHVP

SEQ ID NO: 73 - protein, 47 aa, Saccharomyces cerevisiae
MSDWKVDPDTRRRLLQLQKIGANKKCMDCGAPNPQWATPKFGAFICL

SEQ ID NO: 74 - DNA, 1032 bp, Saccharomyces cerevisiae
atgacaagatctccctggaagcgcctactatggttgaaacaggagtacccagataattatacagatccaagttttattgagttgagagcaagacaaaaggctgagagtaaccagaagtctgatagaaaattatcagaagctgctcgcgctcaaattaggttggattttataagtttctaccaaaccatattgaacacttctttcatttacatcacttttacatatatttactattatggcttcgatcctattccgccaactattttcctttcatttataacattgattatatcaaggacgaaagtcgaccctctattgtcctcattcatggacgtaaagtcttcgctgattatcacatttgcaatgttgactctctctccagtcctcaaatctctttctaaaacaactgcatctgattccatatggacattgtctttttggctgaccctatggtacattttcgttatttcgtcaacaaagtccaaagataaaccctctaacctttccaccaatatacttgtcgcccttgttgctgtcctatcatcgaggctttcgaccacaatcgacgtattctgttttcttttaatttgtattcagttgaatatcattctacccacttatttatcggtgacgaataaggtagtaccaataatttcaaatattattgtatactcatttttgaatgttgctctaggttggatttatatgctgttgattttctttgcttcagtattttatattactgttttacctaagtggttcatctactggaaaatcaattatcataaacgggataacgatctactaagtacatgggatgcaagaacaccaatattggattagtatataggtgcaggcaaataaagagtatcatctttcaaaggggtacgacttctgcccgcaggggcttattgcctgtcctcttcctcgaccccacggtatttcattatatatagagtcactgtaataataattatcatgaaacctgacaagcaaaggcgtcgggcaattaccaaagggcagttcggtcga

SEQ ID NO: 75 - protein, 37 aa, Saccharomyces cerevisiae
MHSHHSHSGDYSAHGTDPLDSVVDQVVNLNFHTYCLT

SEQ ID NO: 76 - protein, 131 aa, Saccharomyces cerevisiae
MGKRVYDPIHDTFQLREDNSDETKADSPMQSVKSGSQEEASPSSIQSETETVTTKSIPVIHEIEIDDKNDDDSTQSEEENTNILLNFEPSTVPEATGASTATGPVTTNTVRRKPKESNASKYNRHLKKPDG

SEQ ID NO: 77 - DNA, 1230 bp, Saccharomyces cerevisiae
atgcagaagagcatattgctgactaaacctgacggaacacaatcgaatctacacagcatcaaaacggaaacgcccaccacggtggagttcgactcggagcagatggaaaggggccacagggaaagaggtcgtagcaagaagaaaagaggcgaacgagactcaaacgtgagcagtctgtcgcggtcgagaagcagggccagtagccgcagcagagtaagggaggaagagttcctcaagtggaccgtgttgaggcaggacccctcgatgcggttgagggtcgtggatgtggattctgaagaggaaggtgagggcaacgacgaagatgacgacgacggcgacggcgacgatatggacgaggaagagtccgatgaagagcaagtgagcgatatagagaacgatttagagattgacgaggagttccactacgatctgggcatgaaagtgttacccaacttttgtaccagcataaacgaagtgctagactccagcaagccctggatagccaagtacgagatcagcatccgtggccacgaaaacgaagacgtgtctctggagcaactcgacggaggctacgtcagagccatgcaactactcaccaagggtgccggcgcagaggccgggaaccaaaggtccttcatcctctacacggacctgagcagcgagtccacctacgccctgacctatctcatgggcgcagctgtcaaccagggagacaccgtctacattgtccactgggagccctcgaagcccacggacgactcccagatgttcgccaacgttgccagaatcagaaagcacgtcatgcacctgtttgactgcgtcgcgggcgtgctcgacgacctcgacgtcgtcgtcctctccttgacccatccgtacccaaaacacctcctcaacgagatgatccacggcctcaagccagtcgccctgtgctgctccctctcggtcatcctgtccactctgcagaacttcgtctgctctgtgcccatcctcgcggttagaaagaagctgaaacgtgccaagcgcaagggcatcagcgagtgatatataggtgcaggcaaataaagagtatcatctttcaaacttatatacagcaacagaccttatgtcttttctctgctccgatacgttatccacccaactttatttcagttttggcagggggaatttcacaaccccgcacgctaaaaattgtatttaaacttaaaaaagaacagccacaaatagggaact

SEQ ID NO: 78 - DNA, 438 bp, Saccharomyces cerevisiae
atgtcaacatatgacgaaatcgaaatcgaagatatgacgtttgagcctgaaaatcaaatgttcacctatccttgtccctgtggagataggtttcaaatatatctggatgacatgtttgagggcgaaaaagttgctgtttgtcccagctgctcactgatgatcgatgtagttttcgataaagaagacttggctgagtactacgaagaggcaggcatccacccccctgagcctattgccgctgctgcctaatatataggtgcaggcaaataaagagtatcatctttcaaaaaagagctatatgacttagcaacgcaagcagaaaagaaggtttgcttcttcgctggactccggttggaattactattcaaaattccaagtgcactgatggaaaacgttttgctcaggttgagctcttttactgcatataaggatactggg

SEQ ID NO: 79 - protein, 47 aa, Saccharomyces cerevisiae
MVQSAVLGFPRIGPNRELKKATEGYWNGKITVDELFKVGKDLRTQNW

SEQ ID NO: 80 - DNA, 777 bp, Saccharomyces cerevisiae
atgattggcagaggtgtgtgctgcagatcgttccacactgctggatctgcctggaagcaatttggatttcccaaaacacaagtgacaacgatttacaacaagactaagagcgcatctaactataaagggtatttaaagcacagagatgctccaggaatgtactatcaaccatcagaatccatcgcaaccggatctgttaacagtgagaccattccacgtagctttatggcagccagtgaccctcgtagagggcttgacatgcctgttcaaagcactaaagcgaagcagtgtccaaatgttctcgtaggtaagagcacagtgaacggcaaaacctatcatctgggacctcaagaaattgatgagatccggaagttacgtcttgacaatcctcaaaagtatacacgcaaatttttggctgcaaaatatggcatttcgccattatttgtatccatggtctcgaaacctagtgaacaacatgtacaaattatggaaagtagattgcaagaaatccaatcacgctggaaggagaagaggcgtatagccagagaggaccgtaagcgtagaaaactcctgtggtaccaggcgtgatatataggtgcaggcaaataaagagtatcatctttcaaactataagtacttcgtattcatatattgatcacaatgcacggagacgcccattaatgtgacttgaatataccacatgaaagctaaccaataagcaatttaaaaagagttaagtacagcttggagacacccaaacgcttctgcttcgacaac

SEQ ID NO: 81 - DNA, 855 bp, Saccharomyces cerevisiae
atgctaccgaccccttatgtaaaatgcgattacgataaagtatatgagcctgctgaggatagcttcctcatactggactgtttggaaaaagagcatgattttttgaaacagaaatttggtaatcgtttggccatcgtttgcgaaattggttcgggatcaggtatcgtcacaacatttctaatgcaaaacaaaataataccgcaggaaaattccatccacttagctgttgatatcaacccatgggcgctcgaagcaactttggatactgcgaagttaaattcatgtaaaagttcttttttagaagtgattcaagctgatttaaactccagtattcggaataatcaggttgatgtcctaatatttaacccaccatatgtgccagcagaatgtgtgccagatgtcccaggatcaagagaggaggcagaccaatggctagatttagcgttattgggtggaaaagacgggatggcgattactgataaactgctacgacaattagaacaaattctttctccagatggtgttgcatatatattattttgtgcaagaaataagcctaaagaagtcattaagaggtttgtcgatacttacaaatggaatgtgaaacttattgaaacgagaaaagcgggatgggaagtcctcagtgtgtacagctttacaaggtgatatataggtgcaggcaaataaagagtatcatctttcaaatctttactcaaagcgtataactatatttcggtttataaagaaaaattcatagctatgctcatgacagaatatcttttttgagatcttcaccacaaccatataaatcaactgaccctagtccgtaaactttctcataatgtagataactca

SEQ ID NO: 82 - protein, 48 aa, Saccharomyces cerevisiae
MVKPIIAPSILASDFANLGCECHKVINAGADWLHIDVMDGHFVPNITL

SEQ ID NO: 83 - DNA, 2112 bp, Saccharomyces cerevisiae
atgctatcgctaaaaaaatcagcgctctccaagttgactttgctcagaaacacaagaacatttacatcgtcagctttggtgcgccaaacgcagggctctgtaaacggttccgcgtccagatctgcagacgggaagtaccacataatagatcacgagtatgactgtgtggtaatcggtgccggtggtgccggccttagagcggcctttggtcttgccgaggcgggctacaagactgcttgtatatccaagcttttccccaccagatcccacactgttgctgctcagggtggtatcaatgccgctctgggaaatatgcacaaggataactggaaatggcatatgtacgatactgtgaaaggatctgattggctaggtgaccaggactccatccattacatgaccagggaagcgcccaaatcgatcattgaactggaacactatggtgttcctttttcaagaactgaaaacggtaagatctaccaaagagcctttggtggtcagaccaaggagtacggtaagggtgctcaggcctatagaacatgcgctgtcgcagacaggacaggacatgctcttttacacacgctttatggccaagctttaagacatgacacacatttctttattgagtactttgccctcgatctgttgacccataatggcgaggtcgttggtgtcatcgcttataatcaggaagacggtaccatccacagattcagagcacacaagaccattattgccactggtggctatggtagagcatacttctcttgtacctctgctcacacatgtacgggtgacggtaatgccatggtttcgcgtgctggtttccccttgcaagatttagagtttgttcaattccatccttcaggtatatatgggtctggttgcttaatcactgaaggtgctcgtggtgaaggtggttttttggttaattctgaaggtgaaagattcatggaacgttacgctcctacggccaaggatctagcttgtagagatgtcgtttccagagcaatcaccatggagatcagagaaggcagaggtgttggtaagaaaaaggaccacatgtacttacaattgagccatctacctccggaagttctaaaggaaagattgccaggtatctctgaaacagcagccatttttgctggtgtagacgtcaccaaggaacctattcccattattcctaccgtccactataacatgggtggtattcccacgaagtggaatggtgaggcattaaccattgatgaagaaactggcgaagacaaggttattcccggtttaatggcttgtggtgaagccgcttgtgtttctgtccatggtgccaatagattaggtgccaattccttgttggatcttgttgtctttggtcgtgctgttgcccatacggttgctgacactttacagcctgggttgccacacaaaccactaccttctgatttgggtaaagaatccatcgcaaacttggataaactaagaaatgctaatggttcaagatctacggcagaaattagaatgaatatgaaacaaactatgcaaaaggatgtttccgtctttagaacacaatcatctttagatgaaggtgttcggaacattactgcagtagagaagacctttgatgatgtgaagacgaccgatagatcaatgatctggaattctgacttggttgaaactctggagctacagaacttattaacctgtgcctcccaaacagctgtttccgctgctaatagaaaggaatcccgtggtgctcatgcaagagaggattatccaaatagagatgacgaacattggatgaagcatacattatcctggcaaaaggacgtcgctgccccagtgactttgaaatacagaagggttatcgatcacactttggacgaaaaggaatgtccttccgtacctccaactgtaagagcctactaatatataggtgcaggcaaataaagagtatcatctttcaaatcttctttaattgcactttttttgtgcgtttgcacttttttaccactgactcactaatttgtatatatacctattaatatacatttacataaagtttcttcttatacatactctatttatttagttatttattaacttactatttattta

SEQ ID NO: 84 - DNA, 1713 bp, Saccharomyces cerevisiae
atgataacaccaagattctctataacacaggatgaggaatttatatttctcaaaatattcataagtaatattaggttcagtgcagtgggtttagagataatcatccaagaaaatatgatcatttttcacttatccccttactatttaagattaagatttcctcacgaattaatcgatgatgagaggtctaccgctcaatacgattccaaagatgaatgcatcaatgtaaaagtagccaaactgaataaaaatgaatatttcgaagatttggacctaccgacaaagttgttggctaggcaaggcgatcttgcaggtgccgatgcattaacagaaaatactgatgcgaaaaaaacccaaaagccccttattcaagaggtcgaaactgatggcgtgtcgaataatattaaagatgatgtgaagactattggtcaaatgggagaggggtttaattgggagatagagcaaaagatggattccagcaccaataatggaatattgaagacaaaatatgggtttgataacttatatgatacagttatatcagtgtcgacaagcaacgggaacgatatcaatgaattagacgatcctgaacacactgacgcgaacgatagagttattgaaaggttaagaaaagaaaatttgaagtttgatcctgagtactatgtttccgagtatatgacacataaatacggtaacgaggaagacttggaaattaacggcattaaagaactactgaagtttacaccttcgatcgtaaagcaatacctgcaatggtacaaagacagtacgaacccaaatctggtcatgccaattgagtttactgacgaggaacaaaaacagatgcaggacaatttacccaagaagtcttatctcgtggaagatataaaaccactttatgtcactatactcagcgttcttttcagttatgtgtttgaacagatcgaaaacgaaggtactcataccacagagagtgcttggaccatgggaaaattgtgccctcaaatatcattcttagaccaacaattaaaacaggtgaacgaactgcaagacggtatgaaagaaatatcaaaggttaataaagacagctctctcatcaagattgcaatcattacaggtatcaggagagccctgagttatccactacatagaaactatgacctagcaatgaaagcgtggacattcgtatattatattctacgaggtggtaagagactagtcataagggcgcttttggacattcatgaaacatttcgtttccatgatgtttactacgtctacgacaaagttctcttggatgatctgactgcgtggttcatttctcagggtagtgagaacgtcataagaagtctggccttggaaatgagaaaagagcaagagtcactttccaagcaggatattgaatttgaatgtattgcatcgttcaacgaacaaactggcgaacctgagtgggagacactaaatataagagagatggaaattttggcagagtctgaataccgagagcagcagcagaacccacaatgatatataggtgcaggcaaataaagagtatcatctttcaaaagagtatatattgattaacgttcctgcaggggcctaaaattgggttacccttctcttcccgcggcttttcccctcatggcattacgtattccatcgaagaagggagaaggctttcaagccttgaagagtgacagcgatccaagcgtgggg

SEQ ID NO: 85 - DNA, 1335 bp, Saccharomyces cerevisiae
atgtcttttagggatgtcctagaaagaggagatgaatttttagaagcctatcccagaagaagccccctttggagatttctttcatacagtacatcattactgaccttcggtgtatcaaaactgcttcttttcacatgctataatgtcaaattgaatggttttgaaaaattagaaactgccttggaacgttccaaaagggaaaatagaggccttatgacggtcatgaaccatatgagtatggtcgatgatccgttagtttgggcaacactaccatataagttatttacgtctttggacaacataagatggtctttgggtgcacataatatttgctttcaaaataaatttctggccaactttttctcacttggccaagtcctttcaacagaaagatttggggtgggcccatttcaaggttctatagatgcttcaataagattgttaagccctgacgacactttagacttggaatggacccctcactctgaggtctcttcttcgctaaaaaaagcctactccccgcccataataaggtcgaagccatcttgggtccatgtttatccagaaggatttgtactacaattatatccgccttttgaaaattcgatgaggtattttaaatggggtattaccagaatgatcctagaagcaacaaagccgcccattgtagtaccaatatttgctacagggtttgaaaaaatagcatccgaagcagtcacagattcaatgtttagacaaattctaccaagaaactttggctctgaaataaatgttaccataggggatcctttaaatgatgatttaatcgacaggtatagaaaagaatggacacatttggttgaaaaatactatgatcccaaaaatcctaacgacctctctgacgaattgaaatatggtaaagaggcgcaagatttaagaagcagattagccgctgaactgagagcccatgttgctgaaattagaaatgaagttcgcaaattaccacgcgaagaccctaggttcaaatccccctcatggtggaagcggttcaacaccacggaaggtaaatcggacccagatgttaaagtcattggcgaaaattgggcaataaggaggatgcaaaagtttctgcctccagagggtaaaccaaagggtaaggatgattgatatataggtgcaggcaaataaagagtatcatctttcaaagaggtcatttttacataacaaatattcattacttactttgctttttatctgtgcgcattaaaatgagtttcatatataatatagttacatgtgatgaatgtatggacagtctctgtcatttgtcaaaggagtgaaaaagaccagaaaagg

SEQ ID NO: 86 - protein, 57 aa, Saccharomyces cerevisiae
MNANIWVAASDGNLDRVEHILRESKGAMTPQSKDINGYTPMHAAAAYGHLDLLKKMC

SEQ ID NO: 87 - DNA, 1731 bp, Saccharomyces cerevisiae
atggcagataaattaatggacaaaaattttgaagaactgtgctattcatgccgcactggtgatatggacaatgttgatagattgatttccacgggagtaaatgtgaatagtgttgacaagtttgacaattcgccactttttctggctagcctttgcgggcatgaagcagtcgtgaaacttcttctacagagaggagccgtctgtgatagagacagatatgaaggagctcgatgtatttatggagcattgaccgatactatcagagatacccttttaagttatgatatatccaaggctgtagatgtcaagcaaccctttgctactcatatctcatcgatgtataatgacgaaggcttccttaaaagagatatcacattcagagtttcgaatggaaaactgttcacagcgcataagttcttattatgtgcaaggagcgaaattctagcagaaaaaatggtgaatgagtgggcaaagcatgaaattgtttctcttgaagtgcgcccagatatatttgacatatttttgaaatttctttacttaattccgatcctgcaccaaattgaaccgggacaatatgaagaacttatagagctatctagtaaatttgatattgagttattgcccgaatttttagacaaagcaaggcataccgccgatcctacggaaaagtcgaggttaatgtcagattaccaatataagtttaccgaggttgcaagaagtcaacttttaatatttgtgaacaattgtatctttagatctactgttgatcttgctaatagtgaacgacgagttttttccttgatgaactgtccagcttatccggatgttcaacttatggtaaaaaaccgaaatggagccattcgaatatacccttgtcacctggctgttttgagccgcgcagaatatttcaaggtgatgttcacaaacaacttcaaggagaaagttacatatatcaaagccaagcacgttacaggaaagtataatagtatcattccacagctgacattacccaattgtgagtttgaggttgccgaaattattctccgctacttatatgcagataatacagacattccttggatgtatgccgtcgatgttttgttacttgccgatatacttttagaagaccgcttaaaaactatcgcttctacaattataacacaatcaaaagagtttattcagcaatacaatgtttttgacgtactctatttatcatgggaaataggagtggaacgcttagaacaatttgcagctaaatttatagctatacatttgcaagagttatataaagatccagagattaaaagggcaattatgttgagttcacaacgaatttctctccgccaggaaactgatactattgaactagtggatgacattaggtactatttgctccgaaaatattcttttgaaccggatgatgtggagctgttcgaaaaccaagacgatttagaatacctaaaacaagtagggtatttggagtacagaaaagatatgggtatgttagataatatcctagctgatctggaacttgatgtataatatataggtgcaggcaaataaagagtatcatctttcaaagcgaacggatgaatattgccaatgactgctgcgcactcttaaaaaatataatgacaattataataagaagtatgatactttttctgtaaaaaatatatatacatatgattcacgtgaaaatttttatatttttaattaaatttttataat

SEQ ID NO: 88 - DNA, 804 bp, Saccharomyces cerevisiae
atgaagttctcttctgttactgctattactctagccaccgttgccaccgttgccactgctaagaagggtgaacatgatttcactaccactttaactttgtcatcggacggtagtttaactactaccacctctactcataccactcacaagtatggtaagttcaacaagacttccaagtccaagaccccaaaccacactggtactcacaagtacggtaagttcaacaagacctccaagtctaagaccccaaaccataccggtactcacaagtatggtaagttcaacaagacttccaagtccaagactccaaaccataccggtactcacaagtacggtaagttcaacaagacctccaaatccaagactccaaaccacactggtactcacaagtacggtaagttcaacaagacctccaagtctaagaccccaaaccataccggtactcacaagtatggtaagttcaacaaaaccaaacatgacactaccacttatggtcctggtgaaaaggcccgtaagaacaatgccgcccctggtccatctaatttcaactccataaaattgtttggtgttaccgctggtagtgctgccgtagccggtgccttattactattataatatataggtgcaggcaaataaagagtatcatctttcaaaattttctccggttttatgttttttaccccccctacccccttcatcctgaagtagtaaattctttttattatactatttcgactactatgtgtttacgtgtgcttgtgcaaaagtgaaatttattgtgattgtgttctttttctctttttt

SEQ ID NO: 89 - protein, 46 aa, Saccharomyces cerevisiae
MIIERWEVKLSKCNQNVGGYSVLSGNLKENIKLGRRAQKYLKELRN

SEQ ID NO: 90 - protein, 84 aa, Saccharomyces cerevisiae
MSATLFNNIELLPPDALFGIKQRYGQDQRATKVDLGIGAYRDDNGKPWVLPSVKAAEKLIHNDSSYNHEYLGITGLPSLTSNAA

SEQ ID NO: 91 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAYDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 92 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHKLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 93 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVRACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 94 - DNA, 1113 bp, Saccharomyces cerevisiae
atgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctcaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactaagcatggtgttgctgctatcaccactactaagggtaacgaacactgcttcgttattctaagaggtggcaagaagggtaccaattacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaatag

SEQ ID NO: 95 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCDIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 96 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPIGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 97 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVFLRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 98 - DNA, 1113 bp, Saccharomyces cerevisiae
atgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctcaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactaagcatggtgttgctgctatcaccactactaagggtaacgaacattgcttcgttattctaagaggtggcaagaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaatag

SEQ ID NO: 99 - protein, 54 aa, Saccharomyces cerevisiae
MVDESDKKQQTIDEQSQISPEKQTPNKKDKKKEEEEQLSEEDAKLKTDLELLVE